(19) United States
(12) Patent Application Publication
(10) Pub. No.: US 2024/0016636 A1
(43) Pub. Date: Jan. 18, 2024

(54) PROGRESSIVELY EXPANDING ANTI-MIGRATION STENT

(71) Applicant: Boston Scientific Scimed, Inc., Maple Grove, MN (US)

(72) Inventors: Aran Murphy, Wexford (IE); Fionn Stapleton, Galway (IE); Ryan Desmond Lynch, Roscommon (IE); John O'Driscoll, Galway (IE); Darren Gerard Curran, Galway (IE); David Collins, Galway (IE)

(73) Assignee: Boston Scientific Scimed, Inc., Maple Grove, MN (US)

(21) Appl. No.: 18/352,073

(22) Filed: Jul. 13, 2023

Related U.S. Application Data
(60) Provisional application No. 63/389,290, filed on Jul. 14, 2022.

(51) Int. Cl. A61F 5/00

(52) U.S. Cl. CPC A61F 5/0076; A61F 2250/0039; A61F 2210/0004

(57) ABSTRACT

An example medical device includes a stent having a radially expanding tubular framework. The radially expanding tubular framework includes a first end region, a second end region, a medial region positioned between the first end region and the second end region, and a lumen extending from the first end region to the second end region. The stent further includes a tubular structure positioned over the medial region, the tubular structure is formed from a bioabsorbable material, and is configured to hold the medial region in a first, compressed configuration. Upon bioabsorption of the tubular structure, the medial region of the tubular framework radially expands to a second, expanded configuration. One of the first end region or the second end region includes a first flange structure.

### CROSS-REFERENCE TO RELATED APPLICATIONS

This application claims the benefit of U.S. Provisional Patent Application Ser. No. 63/389,290 filed on Jul. 14, 2022, the disclosure of which is incorporated herein by reference.

### TECHNICAL FIELD

The present disclosure relates generally to the field of implantable medical devices for adjusting accessibility through a passage of a medical device and related systems and methods. More particularly, the present disclosure relates to devices, systems, and methods for controlling and/or changing a passage using a flow-regulating device such as a lumen-apposing device.

### BACKGROUND

Treatment methods for various medical conditions, such as obesity, diabetes, or duodenal ulcers, involve bypassing the duodenum or restricting flow of materials through the duodenum. If the treatment requires complete bypass of the duodenum, then occlusion (e.g., full occlusion) of the pylorus may be indicated, and an anastomosis may be created, such as between the stomach and the jejunum. A lumen-apposing device may be placed between the stomach and the jejunum to allow for passage of materials (fluid, liquid, chyme, etc.) from the stomach and into the jejunum. One challenge presented by such devices is to prevent migration of the device distally into the small intestine or proximally into the stomach. Thus, there is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a stent having a radially expanding tubular framework. The radially expanding tubular framework may include a radially outward surface, a radially inward surface, a first end region, a second end region, a medial region positioned between the first end region and the second end region, and a lumen extending from the first end region to the second end region. The stent may further include a tubular structure positioned over the medial region, the tubular structure may be configured to hold the medial region in a first, compressed configuration. One of the first end region or the second end region may include a first flange structure.

Alternatively or additionally to any of the embodiments above, the tubular structure may be formed from a bioabsorbable material.

Alternatively or additionally to any of the embodiments above, upon bioabsorption of the tubular structure, the medial region of the tubular framework may radially expand to a second, expanded configuration.

Alternatively or additionally to any of the embodiments above, the expansion of the medial region of the tubular framework may be progressive over a period of time due to the bioabsorption of the tubular structure.

Alternatively or additionally to any of the embodiments above, when the medial region is in the second, expanded configuration, the medial region may be configured to engage with a tissue surface, thereby exerting a radial force to prevent migration of the stent.

Alternatively or additionally to any of the embodiments above, the medial region of the tubular framework may include a first, inner diameter when in the first, compressed configuration and a second, inner diameter when in the second, expanded configuration, wherein the second, inner diameter is greater than the first, inner diameter.

Alternatively or additionally to any of the embodiments above, the second, inner diameter may be 25% greater than the first, inner diameter.

Alternatively or additionally to any of the embodiments above, the second, inner diameter may be 10%-25% greater than the first, inner diameter.

Alternatively or additionally to any of the embodiments above, the radially expanding tubular framework may include a coating applied over the tubular framework.

Alternatively or additionally to any of the embodiments above, the other one of the first end region or the second end region may include a second flange structure.

An example stent may include a radially expanding tubular framework having a radially outward surface, a radially inward surface, a first end region, a second end region, a medial region positioned between the first end region and the second end region, and a lumen extending from the first end region to the second end region. The stent may further include a tubular structure formed from a bioabsorbable material positioned over the medial region, the tubular structure configured to hold the medial region in a first, compressed configuration, wherein upon bioabsorption of the tubular structure, the medial region of the tubular framework radially expands to a second, expanded configuration. The radially expanding tubular framework may include a coating applied over the tubular framework.

Alternatively or additionally to any of the embodiments above, when the medial region is in the second, expanded configuration, the medial region may be configured to engage with a tissue surface, thereby exerting a radial force to prevent migration of the stent.

Alternatively or additionally to any of the embodiments above, the medial region of the tubular framework may include a first, inner diameter when in the first, compressed configuration and a second, inner diameter when in the second, expanded configuration, wherein the second, inner diameter is greater than the first, inner diameter.

Alternatively or additionally to any of the embodiments above, the second, inner diameter may be 25% greater than the first, inner diameter.

Alternatively or additionally to any of the embodiments above, the second, inner diameter may be 10%-25% greater than the first, inner diameter.

Alternatively or additionally to any of the embodiments above, the first end region may include a first flange structure, and the second end region may include a second flange structure.

An example stent may include a radially expanding tubular framework having a first end region, a second end region, a medial region positioned between the first end region and the second end region. The stent may further include a tubular structure formed from a bioabsorbable material positioned over the medial region, the tubular structure configured to hold the medial region in a first, compressed configuration, wherein upon bioabsorption of the tubular structure, the medial region of the tubular framework radially expands to a second, expanded configuration. The expansion of the medial region of the tubular framework may be progressive over a period of time due to the bioaborption of the tubular structure, and the medial region of the tubular framework may include a first, inner diameter when in the first, compressed configuration and a second, inner diameter when in the second, expanded configuration, wherein the second, inner diameter may be greater than the first, inner diameter.

Alternatively or additionally to any of the embodiments above, when the medial region is in the second, expanded configuration, the medial region may be configured to engage with a tissue surface, thereby exerting a radial force to prevent migration of the stent.

Alternatively or additionally to any of the embodiments above, the second, inner diameter may be 25% greater than the first, inner diameter.

Alternatively or additionally to any of the embodiments above, the second, inner diameter may be 10%-25% greater than the first, inner diameter.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a side view of a stent positioned between a stomach and a portion of a small intestine;

FIG. 2 illustrates a cross-section view of the stent positioned between the stomach and the portion of a small intestine taken at line 2-2 of FIG. 1;

FIG. 3 illustrates a side view of an exemplary stent;

FIG. 4 illustrates a perspective view of an exemplary tubular structure;

FIG. 5 illustrates a top view of the exemplary tubular structure of FIG. 4;

FIG. 6 illustrates an exemplary stent including a tubular structure positioned between a gastric wall and a portion of a small intestine;

FIG. 7 illustrates the exemplary stent positioned between the gastric wall and the portion of the small intestine of FIG. 6, wherein an anastomosis has formed;

FIG. 8 illustrates a top view of an exemplary stent including a tubular structure;

FIG. 9 illustrates a side view of the exemplary stent including the tubular structure of FIG. 8;

FIG. 10 illustrates a top view of the exemplary stent of FIG. 8, upon bioabsorption of the tubular structure;

FIG. 11 illustrates a side view of the exemplary stent of FIG. 8, upon bioabsorption of the tubular structure;

FIG. 12 illustrates a side view of an exemplary stent including a tubular structure;

FIG. 13 illustrates a top view of the exemplary stent including the tubular structure of FIG. 12;

FIG. 14 illustrates a side view of the exemplary stent of FIG. 12, upon bioabsorption of the tubular structure; and

FIG. 15 illustrates a top view of the exemplary stent of FIG. 12, upon bioabsorption of the tubular structure.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

In accordance with various principles of the present disclosure, an implantable device may be used to extend across an anatomical structure to control or regulate the size of a passage therethrough. For instance, an implantable device may extend across a body passage or lumen, such terms being used interchangeably herein without intent to limit. The body passage or lumen may include, without limitation, a portion of a passage or lumen, a passage or lumen between anatomical structures (passages, lumens, cavities, organs, etc.), a passage created across apposed tissue walls (such as to create an anastomosis) etc. The device has a passage or lumen (such terms being used interchangeably herein without intent to limit) therethrough which may be used to occlude or block or narrow or close or constrict or regulate or control (such terms and conjugations thereof may be used interchangeably herein without intent to limit) the body passage through which the device is positioned. The device may be considered and referenced as an occlusion or lumen-apposing or anastomosis or flow-regulating or flow-controlling device, and such terms and various other alternatives thereto may be used interchangeably herein without intent to limit.

It will be appreciated that devices, systems, and methods as disclosed herein may be used in endoscopic, laparoscopic, and/or open surgical procedure. Preferably, a medical professional may be able to deliver and/or to remove the device endoscopically. Advantageously, devices and systems disclosed herein may be used in minimally invasive procedures such as natural orifice transluminal endoscopic surgery.

The following detailed description should be read with reference to the drawings in which similar elements in different drawings are numbered the same. The drawings, which are not necessarily to scale, depict illustrative embodiments and are not intended to limit the scope of the invention.

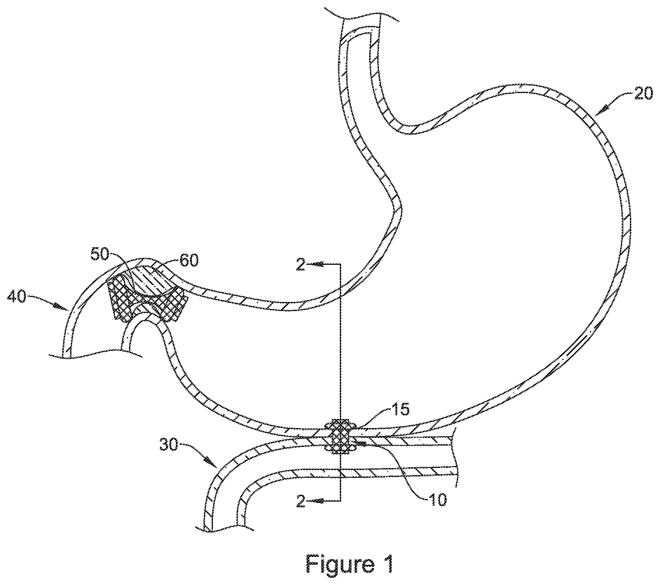

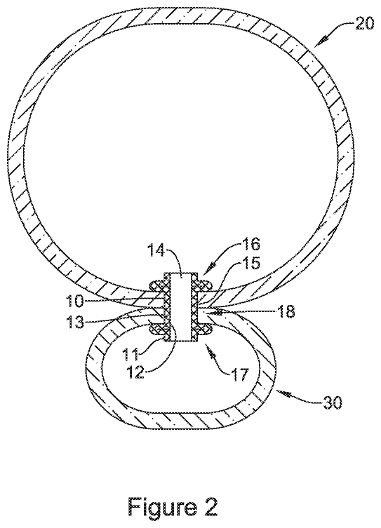

FIG. 1 illustrates a perspective view of an illustrative stent 10 positioned between a stomach 20 and a jejunum 30 (a portion of the small intestine), and FIG. 2 illustrates a cross-section view of the stent 10 positioned between the stomach 20 and the jejunum 30 taken at line 2-2 of FIG. 1. The stent 10 may be a self-expanding stent 10 and may include a radially expanding tubular framework 13 having a radially outward surface 11 and a radially inward surface 12. The radially expanding tubular framework 13 may include a first end region 16, a second end region 17, and a medial region 18 positioned between the first end region 16 and the second end region 17. The radially expanding tubular framework 13 may further include a lumen 14 extending from the first end region 16 to the second end region 17. The stomach 20 normally passes food materials (e.g., chyme, partially digested food materials, fluids, etc.) into a duodenum 40 through a pylorus 60. In some cases, treatment for a patient experiencing obesity, diabetes, or duodenal ulcers, may involve bypassing the duodenum 40, or restricting flow of materials through the duodenum 40. If the treatment requires complete bypass of the duodenum 40, then occlusion (e.g., full occlusion) of the pylorus 60 may be indicated, and an anastomosis 15 may be created between the stomach 20 and the jejunum 30, which may be known as a gastrojejunostomy. FIG. 1 illustrates an example bypass procedure in which a flow restricting device 50 has been positioned within the pylorus 60, thereby restricting access of food materials from the stomach 20 into the duodenum 40 (e.g., a complete bypass). A lumen-apposing metal stent (LAMS), such as the stent 10, may be placed between the stomach 20 and the jejunum 30, thereby forming the anastomosis 15, to allow for passage of food materials (fluid, liquid, chyme, etc.) from the stomach 20 and into the jejunum 30, as shown in FIGS. 1 and 2. While it is illustrated that the stent 10 may be used in forming the anastomosis 15 between the stomach 20 and the jejunum 30, it may be contemplated that the stent 10 may be used to treat a stenosis in a blood vessel, used to maintain a fluid opening or pathway in the vascular, urinary, biliary, tracheobronchial, esophageal or renal tracts, or position a device such as an artificial valve or filter within a body lumen, in some instances. Although illustrated as a stent, the stent 10 may be any of a number of devices that may be introduced endoscopically, subcutaneously, percutaneously or surgically to be positioned within an organ, tissue, or lumen, such as a heart, artery, vein, urethra, esophagus, trachea, bronchus, bile duct, or the like.

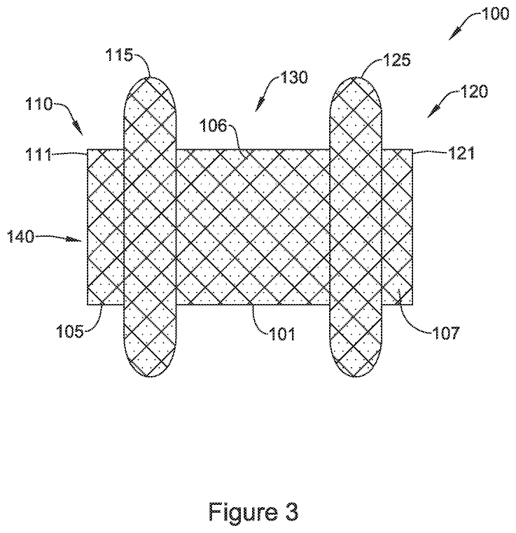

FIG. 3 illustrates a side view of an exemplary stent 100. The stent 100 may be an example of the stent 10 of FIGS. 1 to 2. The stent 100 may include a radially expanding tubular framework 105 having a radially outward surface 101 and a radially inward surface (not shown in FIG. 3). The radially inward surface may be considered as an example of the radially inward surface 12, as shown in FIG. 2. The term ‘radially expanding tubular framework 105’ may be referred to as ‘tubular framework 105’ hereafter. The stent 100 may include a height of 10 millimeters (mm) and an outer diameter (e.g., width) of 20 mm. In some cases, the height of the stent 100 may be 12 mm, 15 mm, 18 mm, between 12 mm and 18 mm, or any other suitable height. In some cases, the outer diameter of the stent 100 may be 18 mm, 22 mm, 25 mm, between 18 mm and 25 mm, or any other suitable diameter. The tubular framework 105 may include a first end region 110, a second end region 120, and a medial region 130 positioned between the first end region 110 and the second end region 120. The tubular framework 105 may further include a lumen 140 extending from the first end region 110 to the second end region 120. The lumen 140 may be considered as an example of the lumen 14, as shown in FIG. 2. In some cases, the first end region 110 may be considered to be a distal end region, and the second end region 120 may be considered to be a proximal end region. In alternative cases, the first end region 110 may be considered to be a proximal end region, and the second end region 120 may be considered to be a distal end region. The first end region 110 may include a first end 111 and the second end region 120 may include a second end 121. The first end region 110 may extend from the first end 111 to the medial region 130, and the second end region 120 may extend from the second end 121 to the medial region 130. The medial region 130 may define a midpoint in the tubular framework 105, such that the first end region 110 and the second end region 120 may have the same lengths. Alternatively, the medial region 130 may be disposed at a location other than a midpoint, such that the first and second end regions 110, 120 have different lengths.

In some cases, the first end region 110 may include a first flange structure 115 and the second end region 120 may include a second flange structure 125. The medial region 130 may be positioned between the first flange structure 115 and the second flange structure 125. The first flange structure 115 and the second flange structure 125 may be considered to be retention members configured to aid in holding the stent 100 in place. Thus, the first and second flange structures 115, 125 may include a width (e.g., an outer diameter) sufficient to provide retention strength. For example, the width of the first and second flange structures 115, 125 may be in the range of 20 to 70 mm. In some cases, the first and second flange structures 115, 125 may include a width greater than that of the first end 111, the second end 121, and the medial region 130 of the tubular framework 105. In some cases, the first and second flange structures 115, 125 may include the same width. In some cases, the first and second flange structures 115, 125 may include differing widths. In some cases, the first and second flanges 115, 125 may include any of a variety of shapes, such as concave, convex, disc-shaped, cylindrical (e.g., having a longer longitudinal extent then illustrated), etc., or other configurations, the particular shape and configuration not being limited by the present disclosure. While it is illustrated that the first flange structure 115 is positioned near the first end region 110 and the second flange structure 125 is positioned near at the second end region 120, it may be contemplated that the first flange structure 115 is positioned near the second end region 120 and the second flange structure 125 is positioned near the first end region 110. In some cases, it may be contemplated that the tubular framework 105 includes only one flange structure (e.g., the first flange structure 115 or the second flange structure 125).

The stent 100 may be configured to be implanted between the stomach and the jejunum of a patient, to form an anastomosis. In other embodiments, the stent 100 may be configured to be implanted in the urinary, biliary, tracheobronchial, esophageal or renal tracts, for example. Since the stent 100, or a portion thereof, may be intended to be implanted permanently in the body lumen, the stent 100 may be made, at least in part, from a biostable material. Examples of the biostable metal materials may include, but are not limited to, stainless steel, tantalum, tungsten, niobium, platinum, nickel-chromium alloys, cobalt-chromium alloys such as Elgiloy® and Phynox®, nitinol (e.g., 55% nickel, 45% titanium), cisplatin, and other alloys based on titanium, including nickel titanium alloys, or other suitable metals, or combinations or alloys thereof. Some suitable biostable polymeric materials include, but are not necessarily limited to, polyamide, polyether block amide, polyethylene, polyethylene terephthalate, polypropylene, polyvinylchloride, polyurethane, polytetrafluoroethylene, polysulfone, and copolymers, blends, mixtures or combinations thereof.

The tubular framework 105 may include a number of interconnected struts 106 to form a mesh-like structure of the tubular framework 105. The struts 106 may be configured to transition from a compressed state to an expanded state. The struts 106 may include a diameter of, for example, 0.0762 mm to 0.3556 mm. The tubular framework 105 may include a coating 107 applied over the struts 106 of the tubular framework 105, thus the entirety of the stent 100 may be covered with the coating 107. The coating 107 may be formed from a silicone and may be configured to prevent leakage of food materials during anastomosis formation. In some cases, the coating 107 may be applied over the struts 106 in the medial region 130. In some cases, the coating 107 may be applied over the struts 106 within the first end region 110 and the medial region 130, and in some cases, the coating 107 may be applied over the struts 106 within the second end region 120 and the medial region 130. These are just examples.

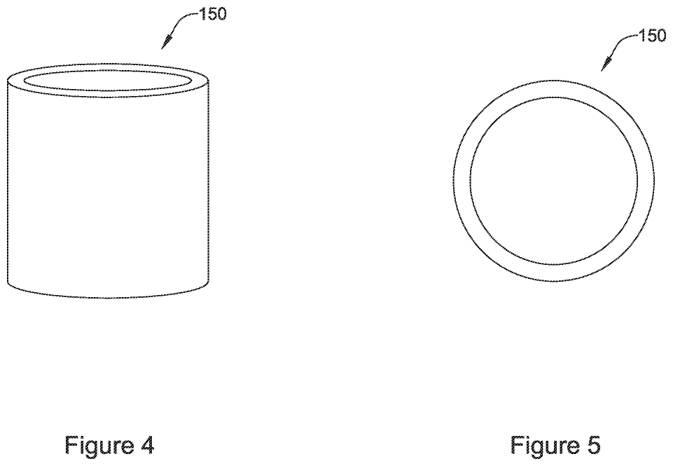

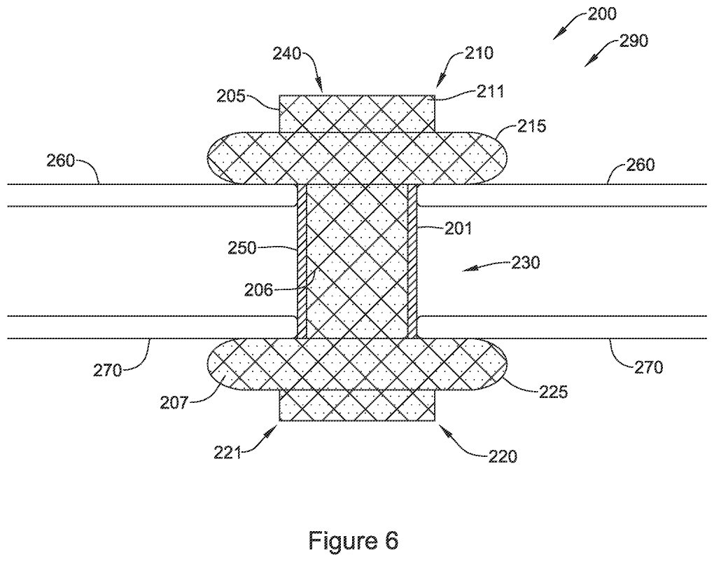

In some cases, the stent 100 may include a tubular structure 150 positioned over the medial region 130, as shown in FIGS. 4 and 5. FIG. 4 illustrates a perspective view of the exemplary tubular structure 150 while FIG. 5 illustrates a top view of the exemplary tubular structure 150. The tubular structure 150 may be formed from a bioabsorbable material, and may be configured to hold the medial region 130 in a first, compressed configuration, as shown in FIGS. 6, 8, 9, 12, and 13. Examples of suitable bioabsorbable materials may include polymers, such as polygycamin, poly-L-lactide (PLLA), polyglycolide (PGA), polylactide (PLA), poly-D-lactide (PDLA), polycaprolactone, polydioxanone, polygluconate, polylactic acid-polyethylene oxide copolymers, modified cellulose, collagen, poly(hydroxybutyrate), polyanhydride, polyphosphoester, poly(amino acids), and combinations thereof.

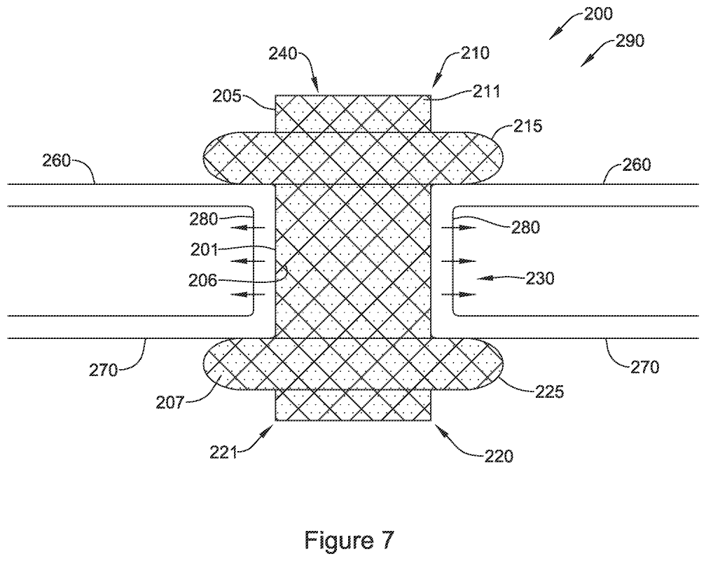

The bioabsorbable material of the tubular structure 150 may be absorbed by the body of a patient through the blood stream, other fluids and/or other natural compositions, over a period of time after implanting the stent 100 within the body. The tubular structure 150 may include a thickness within a range of 0.102 mm to 0.203 mm. In such cases, when the stent 100 is implanted between a gastric wall of the stomach and the jejunum, the bioabsorbable material of the tubular structure 150 may be fully absorbable within six weeks of insertion. In some cases, the tubular structure 150 may have a thickness of 0.05 mm, 0.075 mm, 0.25 mm, 0.30 mm, or any other suitable thickness. In some cases, the bioabsorbable material of the tubular structure 150 may be fully absorbable within two weeks, within four weeks, within eight weeks, or any other suitable time frame. In some cases, the time frame for the bioabsorption of the tubular structure 150 can be adjusted by altering the thickness of the tubular structure 150 and/or by changing the compositions of the tubular structure 150, such as, by including various additives. As previously stated, the tubular structure 150 may be configured to hold the medial region 130 of the tubular framework 105 in a first, compressed configuration. The medial region 130 of the tubular framework 105 may be biased to a second, expanded configuration. Thus, upon bioabsorption of the tubular structure 150, the medial region 130 of the tubular framework 105 may radially expand to the second, expanded configuration, as shown in FIGS. 7, 10, 11, 14, and 15. Thus, the expansion of the medial region 130 of the tubular framework 105 may be progressive over a period of time due to the bioabsorption of the tubular structure 150.

FIG. 6 illustrates an exemplary stent 200 including a tubular structure 250 positioned between a gastric wall 260 (e.g., the stomach) and a portion of a small intestine 270 (e.g., the jejunum). The stent 200 may include a radially expanding tubular framework 205 having a radially outward surface 201 and a radially inward surface (not shown in FIG. 6). The radially inward surface may be considered as an example of the radially inward surface 12, as shown in FIG. 2. The term ‘radially expanding tubular framework 205’ may be referred to as ‘tubular framework 205’ hereafter. The stent 200 may include a height of 10 mm and an outer diameter (e.g., width) of 20 mm. In some cases, the height of the stent 200 may be 12 mm, 15 mm, 18 mm, or any other suitable height. In some cases, the outer diameter of the stent 200 may be 18 mm, 22 mm, 25 mm, or any other suitable diameter. The tubular framework 205 may include a first end region 210, a second end region 220, and a medial region 230 positioned between the first end region 210 and the second end region 220. The tubular framework 205 may further include a lumen 240 extending from the first end region 210 to the second end region 220. The lumen 240 may be considered as an example of the lumen 14, as shown in FIG. 2. In some cases, the first end region 210 may be considered to be a distal end region, and the second end region 220 may be considered to be a proximal end region. In some cases, the first end region 210 may be considered to be a proximal end region, and the second end region 220 may be considered to be a distal end region. The first end region 210 may include a first end 211 and the second end region 220 may include a second end 221. The first end region 210 may extend from the first end 211 to the medial region 230, and the second end region 220 may extend from the second end 221 to the medial region 230. The medial region 230 may include a midpoint in the tubular framework 205, such that the first end region 210 and the second end region 220 may have the same lengths. Alternatively, the medial region 230 may be disposed at a location other than a midpoint, such that the first and second end regions 210, 220 have different lengths.

In some cases, the first end region 210 may include a first flange structure 215 and the second end region 220 may include a second flange structure 225. The medial region 230 may be positioned between the first flange structure 215 and the second flange structure 225. The first flange structure 215 and the second flange structure 225 may be considered to be retention members configured to aid in holding the stent 200 in place. Thus, the first and second flange structures 215, 225 may include a width (e.g., an outer diameter) sufficient to provide retention strength. For example, the width of the first and second flange structures 215, 225 may be in the range of 20 to 70 mm. In some cases, the first and second flange structures 215, 225 may include a width greater than that of the first end 211, the second end 221, and the medial region 230 of the tubular framework 205. In some cases, the first and second flange structures 215, 225 may include the same width. In some cases, the first and second flange structures 215, 225 may include differing widths. In some cases, the first and second flanges 215, 225 may include any of a variety of shapes, such as concave, convex, disc-shaped, cylindrical (e.g., having a longer longitudinal extent then illustrated), etc., or other configurations, the particular shape and configuration not being limited by the present disclosure. While it is illustrated that the first flange structure 215 is positioned near the first end region 210 and the second flange structure 225 is positioned near the second end region 220, it may be contemplated that the first flange structure 215 is positioned near the second end region 220 and the second flange structure 225 is positioned near the first end region 210. In some cases, it may be contemplated that the tubular framework 205 includes only one flange structure (e.g., the first flange structure 215 or the second flange structure 225).

The tubular framework 205 may include a number of interconnected struts 206 to form a mesh-like structure of the tubular framework 205. The struts 206 may be configured to transition from a compressed state to an expanded state. The struts 206 may include a diameter of, for example, 0.0762 mm to 0.3556 mm. The tubular framework 205 may include a coating 207 applied over the struts 206 of the tubular framework 205, thus the entirety of the stent 200 may be covered with the coating 207. The coating 207 may be formed from a silicone and may be configured to prevent leakage of food materials during anastomosis formation. In some cases, the coating 207 may be applied over the struts 206 in the medial region 230. In some cases, the coating 207 may be applied over the struts 206 within the first end region 210 and the medial region 230, and in some cases, the coating 207 may be applied over the struts 206 within the second end region 220 and the medial region 230. These are just examples.

In some cases, the stent 200 may include a tubular structure 250 positioned over the medial region 230, as shown in FIG. 6. In such cases, the medial region 230 of the stent 200 and the tubular structure 250 may be positioned between the stomach and the jejunum, thereby configured to engage with walls of a lumen formed within the stomach and the jejunum. The tubular structure 250 may be formed from a bioabsorbable material, and may be configured to hold the medial region 230 in a first, compressed configuration 290, as shown in FIG. 6. Examples of suitable bioabsorbable materials may include polymers, such as polygycamin, poly-L-lactide (PLLA), polyglycolide (PGA), polylactide (PLA), poly-D-lactide (PDLA), polycaprolactone, polydioxanone, polygluconate, polylactic acid-polyethylene oxide copolymers, modified cellulose, collagen, poly(hydroxybutyrate), polyanhydride, polyphosphoester, poly(amino acids), and combinations thereof.

The bioabsorbable material of the tubular structure 250 may be absorbed by the body of a patient through the blood stream, other fluids and/or other natural compositions, over a period of time after implanting the stent 200 within the body. The tubular structure 250 may include a thickness within a range of 0.102 mm to 0.203 mm. In such cases, when the stent 200 is implanted between a gastric wall of the stomach and the jejunum, the bioabsorbable material of the tubular structure 250 may be fully absorbable within six weeks of insertion. In some cases, the tubular structure 250 may have a thickness of 0.05 mm, 0.075 mm, 0.25 mm, 0.30 mm, or any other suitable thickness. In some cases, the bioabsorbable material of the tubular structure 250 may be fully absorbable within two weeks, within four weeks, within eight weeks, within ten weeks, within twelve weeks, or any other suitable time frame. In some cases, the time frame for the bioabsorption of the tubular structure 250 can be adjusted by altering the thickness of the tubular structure 250 and various additives. As previously stated, the tubular structure 250 may be configured to hold the medial region 230 of the tubular framework 205 in the first, compressed configuration 290. The medial region 230 may include an inner diameter of around 15 millimeters (mm) when in the first, compressed configuration 290. In some cases, the medial region 230 may include an inner diameter of about 10 mm, 12 mm, 18 mm, or any other suitable diameter.

Upon bioabsorption of the tubular structure 250, the medial region 230 of the tubular framework 205 may radially expand to a second, expanded configuration 295, as shown in FIG. 7. The expansion of the medial region 230 of the tubular framework 205 may be progressive over a period of time due to the bioabsorption of the tubular structure 250, allowing for an anastomosis 280 to be formed. The anastomosis 280 forms around the medial region 230 when the medial region 230 is in the first, compressed configuration. Thus, when the tubular structure 250 decays and the medial region 230 is in the second, expanded configuration 295, the medial region 230 may be configured to engage with a tissue surface (e.g., the anastomosis), thereby exerting a radial force to prevent migration of the stent 200, as indicated by the arrows in FIG. 7. In some cases, the progressively larger axial force on the anastomosis 280, may promote a faster patency, thereby reducing migration of the stent 200.

The medial region 230 of the tubular framework 205 may expand by 25% when the bioabsorbable tubular structure 250 absorbs into the body. In some cases, the medial region 230 of the tubular framework 205 may expand by 10% to 25%, or any other suitable percentage. Thus, when the medial region 230 is in the second, expanded configuration, the medial region 230 may include an inner diameter of 20 mm. In some cases, the medial region 230 may include an inner diameter of 12.5 mm, 15 mm, 22.5 mm, between 12 mm and 23 mm, or any other suitable diameter.

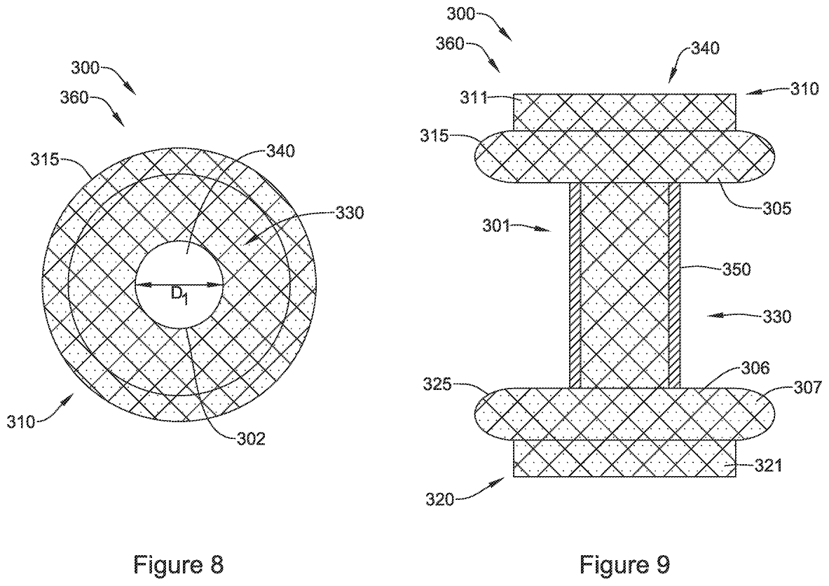

FIGS. 8 and 9 illustrate an exemplary stent 300 in a first, compressed configuration 360, wherein the stent 300 includes a tubular structure 350. The stent 300 may be an example of stent 200 shown in FIGS. 6 and 7. The stent 300 may include a radially expanding tubular framework 305 having a radially outward surface 301 and a radially inward surface 302. The term ‘radially expanding tubular framework 305’ may be referred to as ‘tubular framework 305’ hereafter. The stent 300 may include a height of 10 mm and an outer diameter (e.g., width) of 20 mm. In some cases, the height of the stent 300 may be 12 mm, 15 mm, 18 mm, between 12 mm and 18 mm, or any other suitable height. In some cases, the outer diameter of the stent 300 may be 18 mm, 22 mm, 25 mm, between 18 mm and 25 mm, or any other suitable diameter. The tubular framework 305 may include a first end region 310, a second end region 320, and a medial region 330 positioned between the first end region 310 and the second end region 320. The tubular framework 305 may further include a lumen 340 extending from the first end region 310 to the second end region 320. In some cases, the first end region 310 may be considered to be a distal end region, and the second end region 320 may be considered to be a proximal end region. In some cases, the first end region 310 may be considered to be a proximal end region, and the second end region 320 may be considered to be a distal end region. The first end region 310 may include a first end 311 and the second end region 320 may include a second end 321. The first end region 310 may extend from the first end 311 to the medial region 330, and the second end region 320 may extend from the second end 321 to the medial region 330. The medial region 330 may define a midpoint in the tubular framework 305, such that the first end region 310 and the second end region 320 may have the same lengths. Alternatively, the medial region 330 may be disposed at a location other than a midpoint, such that the first and second end regions 310, 320 have different lengths.

In some cases, the first end region 310 may include a first flange structure 315 and the second end region 320 may include a second flange structure 325. The medial region 330 may be positioned between the first flange structure 315 and the second flange structure 325. The first flange structure 315 and the second flange structure 325 may be considered to be retention members configured to aid in holding the stent 300 in place. Thus, the first and second flange structures 315, 325 may include a width (e.g., an outer diameter) sufficient to provide retention strength. For example, the width of the first and second flange structures 315, 325 may be in the range of 20 to 70 mm. In some cases, the first and second flange structures 315, 325 may include a width greater than that of the first end 311, the second end 321, and the medial region 330 of the tubular framework 305. In some cases, the first and second flange structures 315, 325 may include the same width. In some cases, the first and second flange structures 315, 325 may include differing widths. In some cases, the first and second flanges 315, 325 may include any of a variety of shapes, such as concave, convex, disc-shaped, cylindrical (e.g., having a longer longitudinal extent then illustrated), etc., or other configurations, the particular shape and configuration not being limited by the present disclosure. While it is illustrated that the first flange structure 315 is positioned near the first end region 310 and the second flange structure 325 is positioned near the second end region 320, it may be contemplated that the first flange structure 315 is positioned near the second end region 320 and the second flange structure 325 is positioned near the first end region 310. In some cases, it may be contemplated that the tubular framework 305 includes only one flange structure (e.g., the first flange structure 315 or the second flange structure 325).

The tubular framework 305 may include a number of interconnected struts 306 to form a mesh-like structure of the tubular framework 305. The struts 306 may be configured to transition from a compressed state to an expanded state. The struts 306 may include a diameter of, for example, 0.0762 mm to 0.3556 mm. The tubular framework 305 may include a coating 307 applied over the struts 306 of the tubular framework 305, thus the entirety of the stent 300 may be covered with the coating 307. The coating 307 may be formed from a silicone and may be configured to prevent leakage of food materials during anastomosis formation. In some cases, the coating 307 may be applied over the struts 306 in the medial region 330. In some cases, the coating 307 may be applied over the struts 306 within the first end region 310 and the medial region 330, and in some cases, the coating 307 may be applied over the struts 306 within the second end region 320 and the medial region 330. These are just examples.

As stated above, the stent 300 may include the tubular structure 350 positioned over the medial region 330, as shown in FIG. 9. The tubular structure 350 may be formed from a bioabsorbable material, and may be configured to hold the medial region 330 in the first, compressed configuration 360. When the medial region 330 is in the first, compressed configuration 360, the medial region 330 of a tubular framework 305 may include a first, inner diameter D1 of around 15 mm. In some cases, the medial region 330 may include a first, inner diameter D1 of about 10 mm, 12 mm, 18 mm, between 10 mm and 18 mm, or any other suitable diameter.

The bioabsorbable material of the tubular structure 350 may be absorbed by the body of a patient through the blood stream, other fluids and/or other natural compositions, over a period of time after implanting the stent 300 within the body. The tubular structure 350 may include a thickness within a range of 0.102 mm to 0.203 mm. In such cases, when the stent 300 is implanted between a gastric wall of the stomach and the jejunum, the bioabsorbable material of the tubular structure 350 may be fully absorbable within six weeks of insertion. In some cases, the bioabsorbable material of the tubular structure 350 may be fully absorbable within two weeks, within four weeks, within eight weeks, or any other suitable time frame. In some cases, the time frame for the bioabsorption of the tubular structure 350 can be adjusted by altering the thickness of the tubular structure 350 and various additives.

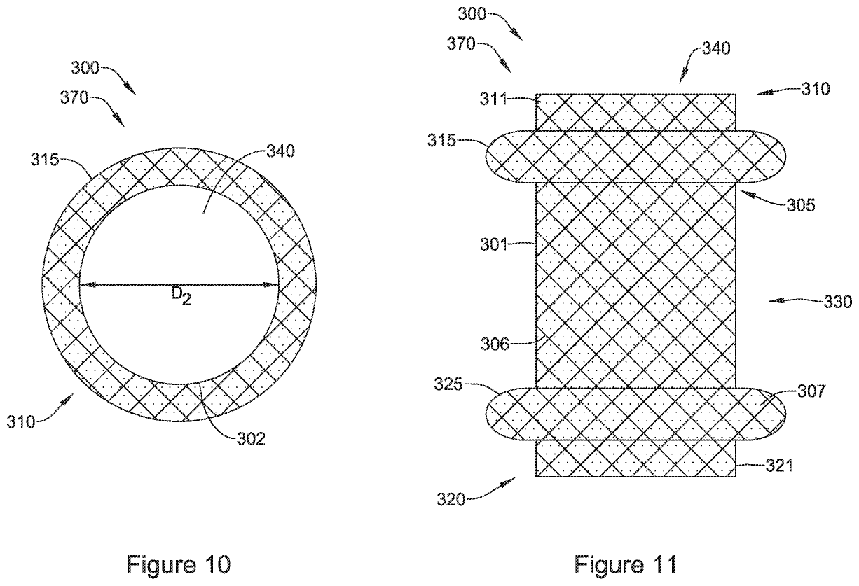

Upon bioabsorption of the tubular structure 350, the medial region 330 of the tubular framework 305 may radially expand to a second, expanded configuration 370, as shown in FIGS. 10-11. The expansion of the medial region 330 of the tubular framework 305 may be progressive over a period of time due to the bioabsorption of the tubular structure 350, allowing for an anastomosis to be formed. The anastomosis forms around the medial region 330 when the medial region 330 is in the first, compressed configuration 360. Thus, when the tubular structure 350 decays and the medial region 330 is in the second, expanded configuration 370, the medial region 330 may be configured to engage with a tissue surface (e.g., the anastomosis), thereby exerting a radial force to prevent migration of the stent 300. In some cases, the progressively larger axial force on the anastomosis, may promote a faster patency, thereby reducing migration of the stent 300.

FIGS. 10 and 11 illustrate the exemplary stent of FIGS. 8 and 9 in the second, expanded configuration 370. When in the second, expanded configuration 370, the medial region 330 of the tubular framework 305 may expand by 25% when the bioabsorbable tubular structure 350 absorbs into the body. In some cases, the medial region 330 of the tubular framework 305 may expand by 10% to 25%, or any other suitable percentage. In other words, the medial region 330 of the tubular framework 305 may include the first, inner diameter D1 when in the first, compressed configuration 360, and a second, inner diameter D2 when in the second, expanded configuration 370. Thus, when the medial region 330 is in the second, expanded configuration 370, the medial region 330 may include the second, inner diameter D2 of 20 mm. In some cases, the medial region 330 may include a second, inner diameter D2 of 12.5 mm, 15 mm, 22.5 mm, between 12 mm and 23 mm, or any other suitable diameter. In some cases, the second, inner diameter D2 may be 25% greater than the first, inner diameter D1. In some cases, the second, inner diameter D2 may be 10% to 25% greater than the first, inner diameter D1. In some cases, the second, inner diameter D2 may be 30% greater than the first, inner diameter D1.

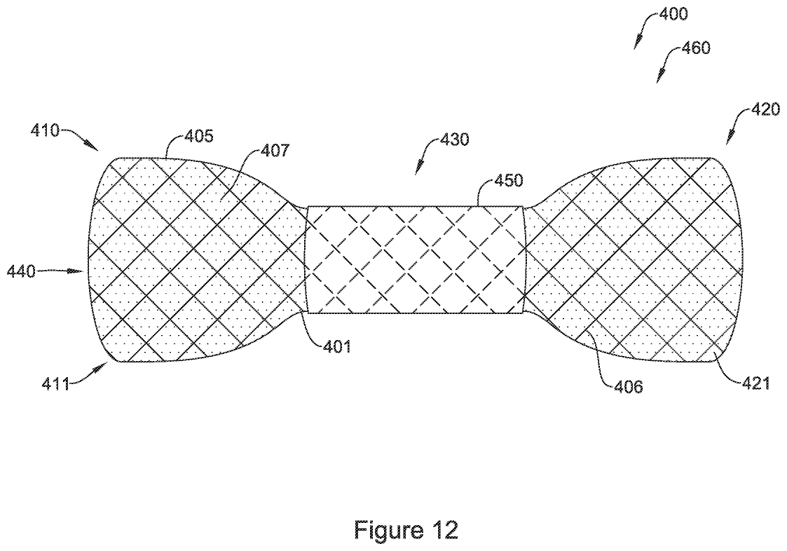

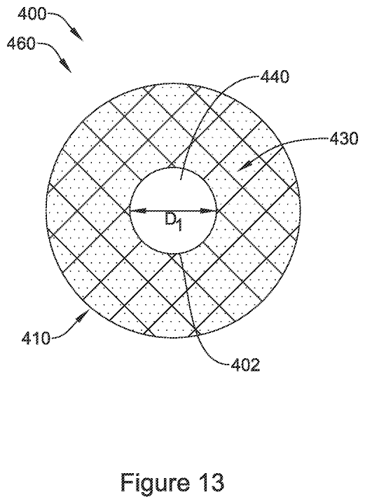

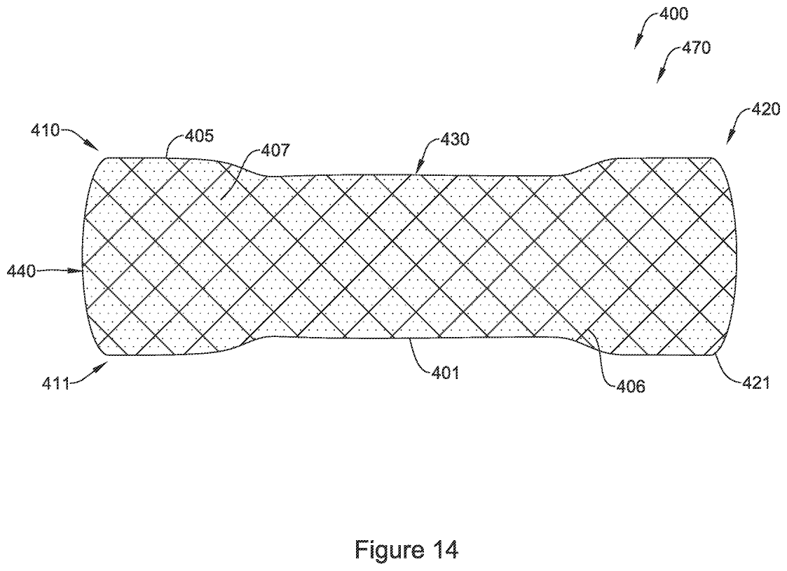

FIGS. 12 and 13 illustrate an exemplary stent 400 in a first, compressed configuration 460, wherein the stent 400 includes a tubular structure 450. The stent 400 may be an example of stent 200 shown in FIGS. 6 and 7. The stent 400 may include a radially expanding tubular framework 405 having a radially outward surface 401 and a radially inward surface 402. The term ‘radially expanding tubular framework 405’ may be referred to as ‘tubular framework 405’ hereafter. The stent 400 may include a height of 10 millimeters (mm) and an outer diameter (e.g., width) of 20 mm. In some cases, the height of the stent 400 may be 12 mm, 15 mm, 18 mm, between 12 mm and 18 mm, or any other suitable height. In some cases, the outer diameter of the stent 400 may be 18 mm, 22 mm, 25 mm, between 18 mm and 25 mm, or any other suitable diameter. The tubular framework 405 may include a first end region 410, a second end region 420, and a medial region 430 positioned between the first end region 410 and the second end region 420. The tubular framework 405 may further include a lumen 440 extending from the first end region 410 to the second end region 420. In some cases, the first end region 410 may be considered to be a distal end region, and the second end region 420 may be considered to be a proximal end region. In some cases, the first end region 410 may be considered to be a proximal end region, and the second end region 420 may be considered to be a distal end region. The first end region 410 may include a first end 411 and the second end region 420 may include a second end 421. The first end region 410 may extend from the first end 411 to the medial region 430, and the second end region 420 may extend from the second end 421 to the medial region 430. The medial region 430 may define a midpoint in the tubular framework 405, such that the first end region 410 and the second end region 420 may have the same lengths. Alternatively, the medial region 430 may be disposed at a location other than a midpoint, such that the first and second end regions 410, 420 have different lengths. In some cases, the first end region 410 and the second end region 420 may each include an outer diameter that is greater than an outer diameter of the medial region 430. As indicated in FIGS. 12 and 14, the stent 400 may include a “bow-tie” shape. Thus, the first end region 410 may gradually increase in diameter from the medial region 430 to the first end 411 and the second end region 420 may gradually increase in diameter from the medial region 430 to the second end 421.

The tubular framework 405 may include a number of interconnected struts 406 to form a mesh-like structure of the tubular framework 405. The struts 406 may be configured to transition from a compressed state to an expanded state. The struts 406 may include a diameter of, for example, 0.0762 mm to 0.3556 mm. The tubular framework 405 may include a coating 407 applied over the struts 406 of the tubular framework 405, thus the entirety of the stent 400 may be covered with the coating 407. The coating 407 may be formed from a silicone and may be configured to prevent leakage of food materials during anastomosis formation. In some cases, the coating 407 may be applied over the struts 406 in the medial region 430. In some cases, the coating 407 may be applied over the struts 406 within the first end region 410 and the medial region 430, and in some cases, the coating 407 may be applied over the struts 406 within the second end region 420 and the medial region 430. These are just examples.

As stated above, the stent 400 may include the tubular structure 450 positioned over the medial region 430, as shown in FIG. 12. The tubular structure 450 may be formed from a bioabsorbable material, and may be configured to hold the medial region 430 in the first, compressed configuration 460. When the medial region 430 is in the first, compressed configuration 460, the medial region 430 of a tubular framework 405 may include a first, inner diameter D1 of around 15 mm. In some cases, the medial region 430 may include a first, inner diameter D1 of about 10 mm, 12 mm, 18 mm, or any other suitable diameter.

The bioabsorbable material of the tubular structure 450 may be absorbed by the body of a patient through the blood stream, other fluids and/or other natural compositions, over a period of time after implanting the stent 400 within the body. The tubular structure 450 may include a thickness within a range of 0.102 mm to 0.203 mm. In such cases, when the stent 400 is implanted between a gastric wall of the stomach and the jejunum, the bioabsorbable material of the tubular structure 450 may be fully absorbable within six weeks of insertion. In some cases, the bioabsorbable material of the tubular structure 450 may be fully absorbable within two weeks, within four weeks, within eight weeks, or any other suitable time frame. In some cases, the time frame for the bioabsorption of the tubular structure 450 can be adjusted by altering the thickness of the tubular structure 450 and various additives.

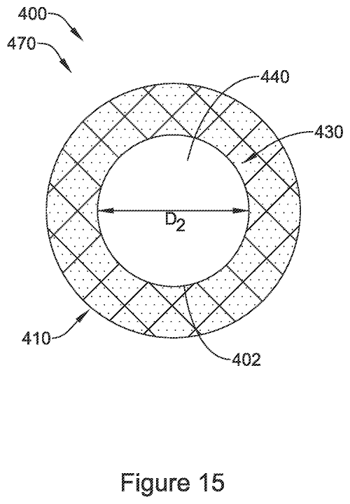

Upon bioabsorption of the tubular structure 450, the medial region 430 of the tubular framework 405 may radially expand to a second, expanded configuration 470, as shown in FIGS. 14 and 15. The expansion of the medial region 430 of the tubular framework 405 may be progressive over a period of time due to the bioabsorption of the tubular structure 450, allowing for an anastomosis to be formed. The anastomosis forms around the medial region 430 when the medial region 430 is in the first, compressed configuration 460. Thus, when the tubular structure 450 decays and the medial region 430 is in the second, expanded configuration 470, the medial region 430 may be configured to engage with a tissue surface (e.g., the anastomosis), thereby exerting a radial force to prevent migration of the stent 400. In some cases, the progressively larger axial force on the anastomosis, may promote a faster patency, thereby reducing migration of the stent 400.

FIGS. 14 and 15 illustrate the exemplary stent of FIGS. 12 and 13 in the second, expanded configuration 470. When in the second, expanded configuration 470, the medial region 430 of the tubular framework 405 may expand by 25% when the bioabsorbable tubular structure 450 absorbs into the body. In some cases, the medial region 430 of the tubular framework 405 may expand by 10% to 25%, or any other suitable percentage. In other words, the medial region 430 of the tubular framework 405 may include the first, inner diameter D1 when in the first, compressed configuration 460, and a second, inner diameter D2 when in the second, expanded configuration 470. Thus, when the medial region 330 is in the second, expanded configuration 470, the medial region 430 may include the second, inner diameter D2 of 20 mm. In some cases, the medial region 430 may include a second, inner diameter D2 of 12.5 mm, 15 mm, 22.5 mm, 12 mm to 23 mm, or any other suitable diameter. In some cases, the second, inner diameter D2 may be 25% greater than the first, inner diameter D1. In some cases, the second, inner diameter D2 may be 10% to 25% greater than the first, inner diameter D1. In some cases, the second, inner diameter D2 may be 30% greater than the first, inner diameter D1.

The stent 10, 100, 200, 300, 400 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of stent 10, 100, 200, 300, 400 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of stent 10, 100, 200, 300, 400 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of stent 10, 100, 200, 300, 400 to achieve the same result.

In some embodiments, a degree of Magnetic Resonance Imaging (MRI) compatibility is imparted into stent 10, 100, 200, 300, 400. For example, stent 10, 100, 200, 300, 400, or portions thereof, may be made of a material that does not substantially distort the image and create substantial artifacts (i.e., gaps in the image). Certain ferromagnetic materials, for example, may not be suitable because they may create artifacts in an MRI image. The stent 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A stent comprising:
a radially expanding tubular framework having a radially outward surface, a radially inward surface, a first end region, a second end region, a medial region positioned between the first end region and the second end region, and a lumen extending from the first end region to the second end region; and
a tubular structure positioned over the medial region, the tubular structure configured to hold the medial region in a first, compressed configuration;
wherein one of the first end region or the second end region includes a first flange structure.

2. The stent of claim 1, wherein the tubular structure is formed from a bioabsorbable material.

3. The stent of claim 2, wherein upon bioabsorption of the tubular structure, the medial region of the radially expanding tubular framework radially expands to a second, expanded configuration.

4. The stent of claim 3, wherein the expansion of the medial region of the radially expanding tubular framework is progressive over a period of time due to the bioabsorption of the tubular structure.

5. The stent of claim 3, wherein when the medial region is in the second, expanded configuration, the medial region is configured to engage with a tissue surface, thereby exerting a radial force to prevent migration of the stent.

6. The stent of claim 3, wherein the medial region of the radially expanding tubular framework includes a first, inner diameter when in the first, compressed configuration and a second, inner diameter when in the second, expanded configuration, wherein the second, inner diameter is greater than the first, inner diameter.

7. The stent of claim 6, wherein the second, inner diameter is 25% greater than the first, inner diameter.

8. The stent of claim 6, wherein the second, inner diameter is 10%-25% greater than the first, inner diameter.

9. The stent of claim 1, wherein the radially expanding tubular framework includes a coating applied over the radially expanding tubular framework.

10. The stent of claim 1, wherein the other one of the first end region or the second end region includes a second flange structure.

11. A stent comprising:
a radially expanding tubular framework having a radially outward surface, a radially inward surface, a first end region, a second end region, a medial region positioned between the first end region and the second end region, and a lumen extending from the first end region to the second end region; and
a tubular structure formed from a bioabsorbable material positioned over the medial region, the tubular structure configured to hold the medial region in a first, compressed configuration;
wherein upon bioabsorption of the tubular structure, the medial region of the radially expanding tubular framework radially expands to a second, expanded configuration; and
wherein the radially expanding tubular framework includes a coating applied over the radially expanding tubular framework.

12. The stent of claim 11, wherein when the medial region is in the second, expanded configuration, the medial region is configured to engage with a tissue surface, thereby exerting a radial force to prevent migration of the stent.

13. The stent of claim 11, wherein the medial region of the radially expanding tubular framework includes a first, inner diameter when in the first, compressed configuration and a second, inner diameter when in the second, expanded configuration, wherein the second, inner diameter is greater than the first, inner diameter.

14. The stent of claim 13, wherein the second, inner diameter is 25% greater than the first, inner diameter.

15. The stent of claim 13, wherein the second, inner diameter is 10%-25% greater than the first, inner diameter.

16. The stent of claim 11, wherein the first end region includes a first flange structure, and the second end region includes a second flange structure.

17. A stent comprising:
a radially expanding tubular framework having a first end region, a second end region, a medial region positioned between the first end region and the second end region; and
a tubular structure formed from a bioabsorbable material positioned over the medial region, the tubular structure configured to hold the medial region in a first, compressed configuration;
wherein upon bioabsorption of the tubular structure, the medial region of the radially expanding tubular framework radially expands to a second, expanded configuration;
wherein the expansion of the medial region of the radially expanding tubular framework is progressive over a period of time due to the bioaborption of the tubular structure; and
wherein the medial region of the radially expanding tubular framework includes a first, inner diameter when in the first, compressed configuration and a second, inner diameter when in the second, expanded configuration, wherein the second, inner diameter is greater than the first, inner diameter.

18. The stent of claim 17, wherein when the medial region is in the second, expanded configuration, the medial region is configured to engage with a tissue surface, thereby exerting a radial force to prevent migration of the stent.

19. The stent of claim 17, wherein the second, inner diameter is 25% greater than the first, inner diameter.

20. The stent of claim 17, wherein the second, inner diameter is 10%-25% greater than the first, inner diameter.
